(19) United States
(12) Patent Application Publication
(10) Pub. No.: US 2004/0147859 A1
(43) Pub. Date: Jul. 29, 2004

(54) Orthopedic cast construction

(76) Inventor: Thomas J O'Keefe, Ann Arbor, MI (US)

Correspondence Address: Benita J Rohm, Rohm & Monsanto, Suite 1525, 660 Woodward Avenue, Detroit, MI, 48226, US

(21) Appl. No.: 10/468,749

(22) PCT Filed: Feb. 25, 2002
(86) PCT No.: PCT/US02/06042

(51) Int. Cl. A61F 5/00

(52) U.S. Cl. 602/3

(57) ABSTRACT

A cast or splint construction that is provided as a flat, multilayered construction pre-configured to have a general form specific to the type of cast to be applied. The multilayer cast construction can be trimmed to size appropriate for the individual body part. The cast construction has one or more inner layers that rest against the patient's skin. Preferably, the skin-contacting layer is made of a material capable of wicking away moisture from the patient's skin. An outer layer of a wettable, breathable, and durable fabric forms the exterior surface of the multilayered cast construction. One or more central layers are sandwiched between the inner layer and the outer layer. The central layers contain one or more layers of padding material and channels of hardenable material. Stitching binds the multiple layers into a unitary multilayer cast construction and defines alternating flexible and hardenable regions. The plurality of alternating flexible and hardenable regions extends in the lengthwise direction relative to the longitudinal axis of a body part, and generally form a ribbed structure. Even after the hardenable regions have been hardened, the flexible regions bend bidirectionally upward and downward in a direction perpendicular to the lengthwise direction of the ribbed structure. This allows for the cast to be removed and re-used during the patient's course of treatment.

### RELATIONSHIP TO OTHER APPLICATION(S)

This application is a continuation of Provisional U.S. Application for Letters Patent, Serial No. 60/270,806 filed Feb. 23, 2001, and claims the benefit thereof.

### BACKGROUND OF THE INVENTION

1. Field of the Invention

The invention generally relates to orthopedic cast or splint construction, and more particularly, to an orthopedic cast or splint that has a unitary, multilayer cast construction.

2. Description of the Related Art

Presently, orthopedic casts are created by applying a series of layered materials to the extremity of a patient. The process typically has three main steps wherein an underlayer of fabric is first applied, followed one or more layers of padding, and then application of an external shell. The underlayer of fabric is typically a cotton gauze-type of material that is somewhat breathable. The purpose of the underlayer is to absorb moisture on the patient's skin to avoid or mitigate against skin irritation under the cast. Unfortunately, cotton gauze is not very practical because is has a tendency to absorb and hold the moisture against the skin, instead of wicking the moisture away from the skin.

The layer of padding typically is formed of a thick cotton material that is rolled on over the underlayer. Several layers of cotton material may be applied in order to achieve the appropriate thickness. The padding layers protects the body part underneath by absorbing any shocks that occur on the surface of the external layer.

The external shell is generally applied in several layers. The external layers are typically constructed of a cotton material dipped in fibreglass or plaster of Paris.

Several layers are generally applied over the padding layers. When the external layers dry, they form a hard, inflexible protective shell that can only be removed by an potentially dangerous, and intrusive procedure, where a cast saw is used to break away the external shell.

Under current practices, casts are removed during a course of treatment to enable the physician to view the injury or to take x-rays in order to check the progress of healing. A new cast must be applied after the x-rays. Another frequent problem with conventional casts is irritation of the skin beneath the cast. When a patient complains of irritation, the cast must be removed in order to look for signs of skin excoriation or irritation. This necessitates the application of a new cast. There is, therefore, a need for a cast construction that will decrease the incidence of skin irritation to the patient.

The problem with removing and applying a new cast is that the new cast can be awkward, or uncomfortable, since the ultimate fit and appearance of the cast is largely dependant upon each individual practitioner's technical ability. Moreover, each time a conventional cast is removed, a cast saw is used which carries a risk of injury to the patient.

There is, therefore, a need for a cast construction that does not require great skill and technical expertise to apply and/or that can be easily removed and reused.

Various approaches have been described in the art for the purpose of providing cast constructions that are reusable and require less skill to apply to a patient. One known arrangement includes a splint made of an envelope of flexible material that has at least two destructible cells of reactants that harden when commingled. When the splint is formed around a body part, the cells holding each of the reactive materials are ruptured and so that the reactants are mixed and the splint will begin to set or harden.

A second approach involves a splint that is made of one or more strips of a multilayer casting material that is an elongated web that can be stored in a roll, like a bandage, and then cut to length from the roll. The surface layers of this known arrangement, which sandwich a central layer, are made of foam, cotton cloth, and a wicking material to aid in setting the splint. The central layer is made up, typically, of several layers of plaster of Paris and woven mesh fabric. Stitching run longitudinally down the middle and sides of the material to help keep the layers together when a length of the material is cut off the main roll. Once the splint material has been selected and cut, it will be soaked in water, wrapped around the desired body portion and held in position until the plaster of Paris hardens.

A third approach involves a variation of the splint described above, wherein the plaster of Paris layer has a layer of thickly padded, wicking material covering at least one side of the layer of plaster of Paris. The wicking material helps to keep the area under the cast free of moisture, which will eliminate some of the unpleasant odor and skin irritations that are associated with a conventional cast. In this known arrangement, the splint material has colored stitching on its surface to indicate to the practitioner which side of the splint has the padding.

One problem with the conventional approaches mentioned above is that they are not flexible once they are mounted to the body part and hardened into a solid mass. Therefore, the known approaches do not permit removal and re-use. Moreover, the approaches mentioned above are not pre-configured for a specific body par and require skill to conform the splint to the body part in a manner that is comfortable to the patient.

### SUMMARY OF THE INVENTION

The foregoing and other disadvantages are overcome by a novel cast construction that is provided as a flat, multilayered construction pre-configured to have a general form specific to the type of cast to be applied. In a first aspect thereof, a multilayered cast construction is provided for use in orthopedic applications. In accordance with the invention, an inner layer forms the interior surface of the multilayered cast construction and is fabricated of a material that is suitable for contact with a human or animal body part. The inner layer, which, is generally defined as the layer closest to the skin, is preferably made of a wicking material. The wicking material can be any known wicking material, such as polypropylene, and includes, but is not limited to, silk, THINSULATE™ or GORTEX™. Of course, the inner layer can comprises multiple layers of material which may, or may not be, wicking material.

An outer layer is formed of a material suitable for forming an exterior surface of the multilayered cast construction. The outer layer is illustratively cotton, nylon, or rayon. However, other flexible materials are acceptable. Some of the characteristics of materials suitable for the outer layer material include materials that are non-irritating, durable, wettable, and breathable. Of course, the outer layer can itself be formed of multiple layers of such materials.

One or more central layers are sandwiched between the inner layer and the outer layer. The central layers of the multilayered cast construction contain one or more layers of padding material and channels of hardenable material. The one or more layers of padding material can be formed of foam padding, fleece, or any suitable shock-absorbent material.

The inner layer, outer layer, and the central layers collectively form the multilayered cast construction which is generally configured to the shape of a body part and has a lengthwise direction parallel to the longitudinal axis of the body part. The multilayered cast construction terminates in the lengthwise direction in a first longitudinal edge and an opposing second longitudinal edge. All the layers form alternating flexible regions of one or more layers of a padding, and adjacent hardenable regions formed of a channel of a hardenable material sandwiched between the one or more layers of a padding material. The plurality of alternating flexible and hardenable regions extend in the lengthwise direction, and generally form a ribbed structure.

In a specific example, the body part is an arm of a human. Illustratively, the multilayered cast construction is pre-configured to be an arm cast. In a further illustrative embodiment, the multilayered cast construction is pre-configured to be a leg cast. In addition, the forms could be sized (i.e., small, medium or large) in shape and design for each of the various casts that are commonly applied in orthopedic practice. Illustratively, short arm casts, long arm casts, short leg casts, long leg casts, and long leg splints would all have their own individual and easily recognizable form and would be provided individually in prepackaged form. The appropriate form would be chosen by the practitioner, wrapped around the extremity, and trimmed with scissors, for example, to adjust for individual variations in size and shape.

In preferred embodiments the channels of hardenable material generally will have a uniform size or shape. However in a further embodiment of the present invention, the channels of hardenable material could vary in size and shape in different regions of the cast. Illustratively, in an arm cast embodiment, the channels of hardenable material could be fabricated to be wider and thicker toward the elbow and narrower near the patient's wrist.

In a preferred embodiment, the channel of hardenable material is a channel of plaster of Paris. In another preferred embodiment, the channel of hardenable material is a channel of fibreglass. The channels of fibreglass or plaster of Paris harden after coming into contact with a reactant, which in these embodiments, is water. Once the cast construction has hardened, the channels of hardenable material form stiff ribs that immobilize the extremity.

The flexible regions bend bidirectionally in a direction perpendicular to the lengthwise direction of the ribbed structure. The flexible regions are regions that do not contain a channel of hardenable material. The ribbed construction, with adjacent flexible regions provide sufficient flexibility for the cast to be removed, and re-applied if required during a course of treatment.

Multilayered cast construction preferably has one or more fasteners attached to the outer layer and extending perpendicular to the first and/or the second longitudinal edges. Illustratively, fastening means, such as Velcro brand hook and loop fastening tape, is provided on at least one longitudinal edge of the cast form so that the first and second longitudinal edges of the cast can be held together. Of course, other fastening means can be used, and it is specifically contemplated that, in some embodiments, the fastening means would be difficult for the patient to release. Illustratively, a more permanent fastening means could be a locking loop fastener.

In a preferred embodiment of the present invention, the multilayered cast construction uses stitching to define the flexible and hardenable regions. In a specific embodiment to the multilayered cast construction a stitched seam on either side of the channel of hardenable material causing the multilayered cast construction to be longitudinally fluted. Additionally the stitched seam will help to keep the multilayered cast construction together as a unitary body during fabrication, packaging, and during the process of casting the invention to the patient's body part. Of course, it is contemplated that the regions can be separated by other means in view of stitching, such as gluing, or depending on the composition of the middle layers by hot weld.

This particular seam is created when heat is applied to a thermoset padding material, so the padding material will change physical form when heat is introduced. In this embodiment the seam is created by applying adhesive and pressure at the location of where the seam is to be created.

In accordance with a method aspect of the invention there is provided the steps of applying the multilayered cast construction of the present invention as follows:

sizing a multilayered cast construction to a surface that the multilayered cast construction is to be applied by trimming the multilayered cast construction with a cutting instrument;

moistening the trimmed multilayered cast construction in a reactant that will react with the hardenable material found in the hardenable channels of the multilayered cast construction;

removing, such as by wringing out, the excess reactant, if necessary;

mounting the multilayered cast construction over a body part; and

hardening the multilayered cast construction by allowing it to dry.

Of course, the method may also include the further step of fastening the multilayered cast construction to the body part with a fastener either or both before or after the step of hardening. This step allows the multilayered cast construction to be more securely held onto the patient's body part as the body part is moved in different directions.

In yet another embodiment of the present method aspect of the invention includes the step of initially wrapping a flexible elastic bandage, illustratively an Ace™ bandage over the mounted multilayered cast construction to aid in molding the multilayered cast construction during the hardening step. Applying a bandage during the hardening step contributes two-fold to the method by (1) holding the multilayered cast construction to the body part since it can slip off prior to hardening and (2) by pressing the cast construction firmly against the patient's skin so that the cast conforms to the contours of the body part.

The advantages of the multilayered cast construction of the present invention are numerous. A high-quality cast can be applied in most circumstances by an individual with limited training. Since the cast is flexible, it may be removed without the use of a cast saw and re-used again if further treatment is needed. This decreases the likelihood of injuring the patient during cast removal and also allows the treating physician to view an operative wound without the need to reapply a new cast. This design will save time in the application process of an initial cast and will decrease the number of casts needed for treatment of a specific fracture because it is removable and reusable. Moreover, if a patient complains about irritation, the cast can be easily and quickly removed, padding can be applied where appropriate, and the same cast can be re-applied.

### BRIEF DESCRIPTION OF THE DRAWING

Comprehension of the invention is facilitated by reading the following detailed description, in conjunction with the annexed drawing, in which:

FIG. 1 is a plan view of a short arm cast embodiment;

FIG. 2 is a cross-sectional view of cast material of the preferred embodiment;

FIG. 3 is a perspective view of an arm cast embodiment set to the arm of a patient; and

FIG. 4 is a plan view of a short leg cast embodiment.

### DETAILED DESCRIPTION OF THE INVENTION

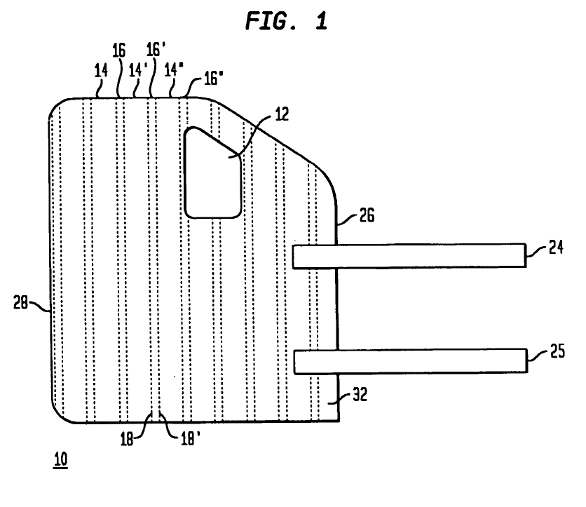

FIG. 1 is a plan view of a short arm cast embodiment of an orthopedic multilayer cast construction, in accordance with the present invention, as shown laying flat so that the exterior layer, or outer layer 32, is visible. In accordance with this specific illustrative embodiment, a pre-configured arm cast 10 has a thumb hole 12 pre-cut into the cast so that it can be wrapped around the hand and arm of a patient (not shown).

Arm cast 10 has a plurality of hardenable regions, illustratively hardenable regions 14, 14′, and 14″, referred to herein collectively as hardenable regions 14, that can be made rigid as will be described in detail hereinbelow. The hardenable regions alternate with adjacent flexible regions, illustratively regions 16, 16′, and 16″, referred to herein collectively as flexible regions 16. In this preferred embodiment, stitching, shown illustratively as seams 18 and 18′, separates and defines hardenable regions 14 and flexible regions 16 so that the multilayer cast construction will be sturdy and will remain flexible even after the cast has been hardened. One or more fasteners, illustratively, hook and loop (i.e., Velcro brand) fasteners 24 and 25, are used to secure a first longitudinal edge 26 of arm cast 10 to the second longitudinal edge 28. Fasteners 24 and 25 may be used to hold the multilayer cast construction in place both before and after hardening forms a rigid cast around the patient's body part (not shown).

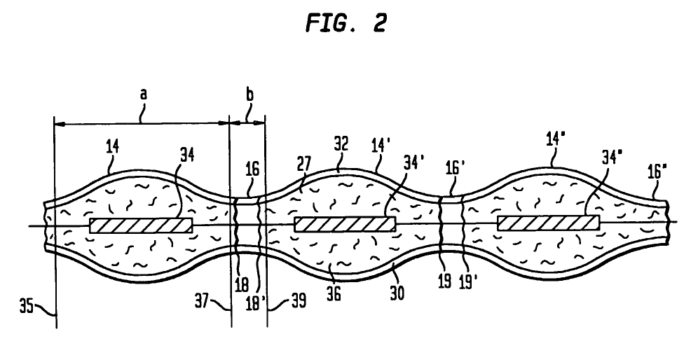

FIG. 2 is a cross-sectional view of the multilayered cast construction in accordance with the present invention. Elements of structure that are identical to those in FIG. 1 are similarly designated in FIG. 2. FIG. 2 shows hardenable regions 14, 14′, and 14″ adjacent to corresponding flexible regions 16, 16′, and 16″. More specifically, hardenable region 14 is defined as generally the region “a” between vertical lines 35 and 37. Flexible region 16 is defined as generally the region “b” between vertical lines 37 and 39. The stitching, shown here specifically as stitched seams 18 and 18″, defines flexible region 16 and separates hardenable region 14 from the hardenable region 14′.

Hardenable region 14 comprises one or more central layers, illustratively central layers 36 and 27 of a flexible, padding material, sandwiched between outer layer 32 and inner layer 30. Channels of a hardenable material, such as plaster of Paris or fibreglass, illustratively channels 34, 34′, 34″, referred to herein collectively as channels 34, are disposed in the center of hardenable regions 14 and, in this embodiment, between layers 36 and 27. Channels 34, once hardened, form a rigid ribbed structure that keep the body part (not shown in this figure) protected and immobile. In an illustrative embodiment, the flexible padding material used to form layers 36 and 27 is a foam material. The padding material of layers 36 and 27 absorbs shock from the outside environment and protects the body part from discomfort and irritation by the hardened ribs. Inner layer 30 forms the inside of the cast and will contact the patient's skin. Therefore, inner layer 30 is preferably formed of a material that is capable of wicking away perspiration and other moisture from the patient's skin in order to decrease discomfort. Outer layer 32 forms the outside of the cast will be exposed to the outside environment. Therefore, outer layer 32 is preferably constructed of a material that is durable enough to withstand the wear and tear of the outside environment and to protect the underlying layers. In certain embodiments of the invention, outer layer 32 may serve as additional padding to absorb any shock that may be introduced from the outside environment.

In a practical embodiment, the multilayer cast construction is constructed as a unitary body such that inner layer 30, central layers 36 and 27, and outer layer 32 are continuous layers that are used to define both the plurality of hardenable regions 14 and the plurality of flexible regions 16. Stitching, illustratively stitched seams 18, 18′, 19 and 19′, penetrates through outer layer 32, central layers 36 and 27, and inner layer 30, to form the unitary construction.

The stitching extends longitudinally on the cast construction, in the direction of the vertical axis of the body part (not shown) for which the cast is intended (see, FIG. 1). As is evident from FIG. 2, stitched seams 18, 18′, 19 and 19′ form the boundaries between hardenable regions 14 and flexible regions 16 by creating a barrier between the two regions. As a result, the longitudinal stitches give arm cast 10 a fluted appearance (see, FIG. 2). The stitching holds the multiple layers of the cast construction together even after cast has been trimmed, soaked and allowed to harden.

Most importantly, the longitudinal flexible regions 16 defined by the stitching make the cast flexible and reusable. Although not specifically shown in the drawing, the periphery of a pre-configured multilayer cast construction can be bound as is known in the art.

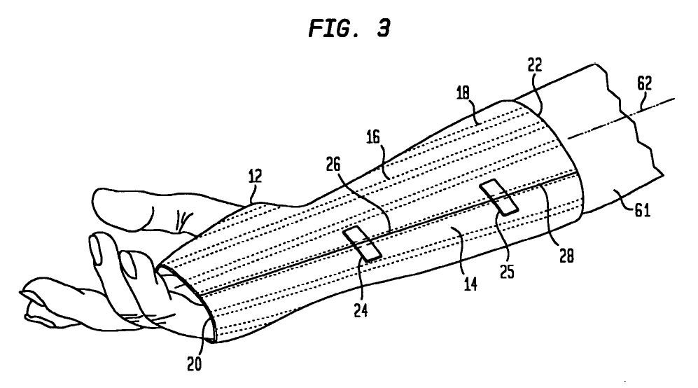

FIG. 3 is a perspective view of the arm cast embodiment of FIG. 1 mounted to an arm of a patient. Elements of structure that are identical to those in FIG. 1 are similarly designated in FIG. 3. This particular embodiment has thumb hole 12 so that the multilayered cast construction can be applied circumferentially around the patient's hand and arm 38. Prior to hardening the cast, the pre-configured cast construction can be can be shortened by trimming, as necessary, at either one, or both, of first lateral edge 20 or second lateral edge 22. The cast may also be trimmed, as necessary, at either, or both, longitudinal edges 26 and 28, so as to surround arm 38 completely, and in this embodiment, without significant overlap. Fasteners 24 and 25 hold longitudinal edges 26 and 28 together. As shown, hardenable regions 14, once reacted to become rigid, form stiff ribs, running in the direction of longitudinal axis 62 of arm 38 and immobilize the patient's arm. During a course of treatment, arm cast 10 can be removed by extending flexible regions 16 in a direction to unwrap the hardened cast and replaced.

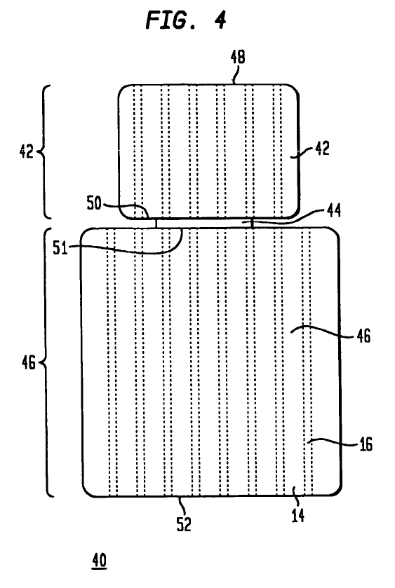

Although the invention has been described hereinabove as a short arm cast embodiment, it is to be understood that this invention is not limited to any particular form of cast or splint, and may be implemented in a wide variety of embodiments, including and certainly not limited to, a leg cast, a neck cast, or a torso cast. Of course, the invention would find application in the veterinary field. In this regard, FIG. 4 illustrates a short leg cast embodiment in accordance with the present invention.

Referring to FIG. 4, a multilayered cast construction, in the form of leg cast 40, is configured generally to have a shape suitable for conforming to a patient's leg, ankle, and foot (not shown). Leg cast 40 is provided with generally rectangular foot portion 42, ankle portion 44, and leg portion 46. Foot portion 42 terminates in lateral edge 48 at the toe, for example. Ankle portion 44 is connected to an opposing lateral edge 50 of foot portion 42. Ankle portion 44 has a generally rectangular shape and is narrower in width than foot portion 42 or leg portion 46. Ankle portion 44 connects to leg portion 46 at lateral edge 51. Leg portion 44 terminates in lateral edge 52 at the calf, for example.

In a method of use, all portions of leg cast 40 will be wrapped around the top of the respective body parts and may be trimmed to a width suitable to surround completely the patient's body parts. If desired, the length of one or more of portions 42 or 46, may be trimmed to terminate at user-selectable point(s), illustratively just above the toes and mid-calf. The trimmed multilayer cast construction is then wetted with a suitable reactant to cause hardenable regions 14 to become rigid. In this manner, hardenable regions 14 of each portion of leg cast 10, form rigid ribs that extend in a lengthwise direction parallel to the vertical axis of the respective body part (i.e., foot, ankle and leg) for which they are intended to protect.

Although the invention has been described in terms of specific embodiments and applications, persons skilled in the art can, in light of this teaching, generate additional embodiments without exceeding the scope or departing from the spirit of the claimed invention. Accordingly, it is to be understood that the drawing and description in this disclosure are proffered to facilitate comprehension of the invention and should not be construed to limit the scope thereof.

What is claimed is:

1. A multilayered cast construction for applying to a body part comprising:
an inner layer forming an interior surface of the multilayered cast construction;
an outer layer of a material suitable for forming an exterior surface of the multilayered cast construction; and
one or more central layers sandwiched between said inner layer and said outer layer, wherein the central layers form alternating flexible regions of one or more layers of a padding material and adjacent hardenable regions formed of a channel of a hardenable material sandwiched between the one or more layers of a padding material; the inner layer, outer layer and central layers form the multilayered cast construction which is generally configured to the shape of a body part and has a lengthwise direction parallel to a vertical axis of the body part, the multilayered cast construction terminates in the lengthwise direction in a first longitudinal edge and an opposing second longitudinal edge; the plurality of alternating flexible and hardenable regions, extend in the lengthwise direction to form a ribbed structure.

2. The multilayered cast construction of claim 1 wherein the flexible regions bend bidirectionally upward and downward in a direction perpendicular to the lengthwise direction of the ribbed structure.

3. The multilayered cast construction of claim 1 wherein the multilayered cast construction is pre-configured to be an arm cast.

4. The multilayered cast construction of claim 1 wherein the multilayered cast construction is pre-configured to be a leg cast.

5. The multilayered cast construction of claim 1 wherein the multilayered cast construction is pre-configured to be a torso cast.

6. The multilayered cast construction of claim 1 wherein the inner layer comprises a layer of polypropylene that wicks away moisture.

7. The multilayered cast construction of claim 1 the one or more layers of padding comprises a foam padding.

8. The multilayered cast construction of claim 1 wherein the one or more layers of padding are comprised of fleece.

9. The multilayered cast construction of claim 1 wherein the channel of hardenable material is a channel of plaster of Paris.

10. The multilayered cast construction of claim 1 wherein the channel of hardenable material is a channel of fibreglass.

11. The multilayered cast construction of claim 1 further comprising one or more fasteners attached to the outer layer of the apparatus and extending perpendicular to the first and the second longitudinal edges, wherein the one or more fasteners are arranged to be connected between the first and second longitudinal edges.

12. The multilayered cast construction of claim 11 wherein the one or more fasteners are Velcro™ strips connectable to the top layer of the apparatus.

13. The multilayered cast construction of claim 1 further comprising a stitched seam on either side of each of the channel of hardenable material for producing a longitudinal flute in the multilayered cast construction.

14. The multilayered cast construction of claim 14 wherein the stitched seam is a hot welded seam.

15. The multilayered cast construction of claim 14 wherein the stitched seam is a glued seam.

16. A method of applying a multilayered cast construction comprising the steps of:
sizing a multilayered cast construction to a body part by trimming the multilayered cast construction with a cutting instrument;
moistening the trimmed multilayered cast construction in a reactant that will react with a channel of hardenable material located in the multilayered cast construction;
wringing out the excess reactant;
mounting the multilayered cast construction over a body part; and
hardening the cast construction by allowing it to dry.

17. The method of claim 13 further comprising the step of fastening the multilayered cast construction to the body part with a fastener.

18. The method of claim 13 further comprising the step of initially wrapping an Ace brand bandage over the mounted multilayered cast construction to aid in molding the multilayered cast construction during the hardening step.
